(19) United States
(12) Patent Application Publication
(10) Pub. No.: US 2021/0052782 A1
(43) Pub. Date: Feb. 25, 2021

(54) ELECTROSPUN-COATED MEDICAL DEVICES

(71) Applicants: TECHNION RESEARCH & DEVELOPMENT FOUNDATION LIMITED, Haifa (IL); HEALTH CORPORATION OF GALILEE MEDICAL CENTER, Nahariya (IL)

(72) Inventors: Eyal ZUSSMAN, Haifa (IL); Aiman ABU AMMAR, Baqa Algharbiya (IL); Maayan GRUBER, Tal-El (IL)

(21) Appl. No.: 16/962,009

(22) PCT Filed: Jan. 15, 2019
(86) PCT No.: PCT/IL2019/050057

§ 371 Date: Jul. 14, 2020

Related U.S. Application Data
(60) Provisional application No. 62/617,369, filed on Jan. 15, 2018.

(51) Int. Cl. A61L 31/10; A61L 31/04; A61L 31/14; A61L 31/16; A61M 16/04

(52) U.S. Cl. CPC A61L 31/10; A61L 31/043; A61L 31/148; A61L 31/16; A61M 16/04; A61L 2300/404; A61M 2207/10; A61L 2300/416; A61L 2300/42; A61L 2300/43; A61L 2300/604; A61L 2300/802; A61L 2400/12; A61L 2300/41

(57) ABSTRACT

Compositions comprising electrospun fibers and pharmaceutical agents encapsulated thereto are provided. Further, articles such as medical devices and methods of use of said fibers, including, but not limited to coating of medical tubes, are provided.

### CROSS REFERENCE TO RELATED APPLICATIONS

This application claims the benefit of priority of U.S. Provisional Patent Application No. 62/617,369, filed Jan. 15, 2018, and entitled: “ELECTROSPUN-COATED MEDICAL DEVICES”, the contents of which are incorporated herein by reference in their entirety.

### FIELD OF INVENTION

The present invention is in the field of medical devices.

### BACKGROUND OF THE INVENTION

Medical devices, such as tubes and catheters are typically used by directly contacting the mucosa and other tissues or organs of the body, for prolonged time periods. This may lead to an inflammation and/or infectious reaction by the body to these devices and may frequently progresses into a pathological state. This reaction is seen in the use of various tubes or implants, such as, endotracheal tubes (ETT), ear ventilation (PE) tubes, gastrostomy tubes, foley catheters, surgical drains, intravenous catheters, cochlear implants and the like.

As a non-limiting example, endotracheal intubation is a widely used technique to support ventilation in the operating room and intensive care units. Pressure exerted by endotracheal tubes typically deployed in airway management, can result in laryngeal and tracheal morbidity. Among the complications are sore throat, cough, hoarseness, dyspnea and post-extubation stridor, due to local irritation and inflammation. Post-extubation upper airway obstruction is another possible complication of both pediatric and adult mechanical ventilation.

The common clinical approach to prevent post-extubation complications and reintubation is the use of multiple steroid intravenous (IV) doses over hours to days prior to elective extubation. Mainly high-risk groups may benefit from this intervention. Reintubation as a result of laryngeal injury or increased length of stay for additional monitoring and treatment likely contributes to hundreds of millions of dollars in healthcare costs each year. On average, patients who are re-intubated in an Intensive Care Unit setup have five additional days of mechanical ventilation at a cost of thousands of dollars per day. Recently, meta-analysis of 18 randomized controlled trials indicated that prophylactic administration of corticosteroids is effective in decreasing the frequency and harshness of postoperative sore throat and hoarseness, as well as the incidence of laryngeal edema and reintubation.

### SUMMARY OF THE INVENTION

According to an aspect of some embodiments of the present invention there is provided a medical device at least partially coated by a composition comprising an electrospun biodegradable nanofiber and at least one active agent, the active agent being encapsulated within the electrospun biodegradable nanofiber, so to locally and sustainably release the active agent.

In some embodiments, the biodegradable nanofiber comprises a polymer or copolymer selected from a miscible polymer, and an enzymatic-degradable polymer.

In some embodiments, the nanofiber is characterized by an adhesion force of 0.4-0.8N to an exterior surface of said medical device.

In some embodiments, the nanofiber has a Young's Modulus in the range of 10-145 MPa.

In some embodiments, the nanofiber has a tensile strength in a range of 0.2-2 MPa.

In some embodiments, the nanofiber has a diameter in the range of 500-1500 nm.

In some embodiments, the nanofiber has a Fibrous Mesh porosity of 78-to 92%

In some embodiments, the nanofiber has a Fibrous Mesh pore diameter 5-15 μm

In some embodiments, the coating comprises a substantially uniform thickness in the range of 150-300 μm.

In some embodiments, the medical device has an agent-loading capacity of: 50-500 μg/cm; and/or 100-1000 μg/cm2 fiber.

In some embodiments, the sustainable release of the active agent is for at least 24 hours.

In some embodiments, the electrospun nanofiber comprises a polymer selected from the group consisting of poly (lactic-co-glycolic) acid (PLGA), polylactic acid (PLA), polyglycolic acid (PGA), and polycaprolactone (PCL).

In some embodiments, the agent is selected from the group consisting of: an anti-inflammatory agent (e.g., steroid), an anti-infective agent (e.g. antibiotics, antifungals), compounds that reduce surface tension (e.g. surfactant), anti-neoplastic agents and anti-proliferative agents, anti-thrombogenic and anticoagulant agents, antiplatelet agents, hormonal agents, nonsteroidal anti-inflammatory drugs (NSAIDs), antimitotics (cytotoxic agents) and antimetabolites.

In some embodiments, the medical device is a tracheal tube. In some embodiments, the agent is an anti-inflammatory agent. In some embodiments, the medical device is a tracheal tube and the agent is an anti-inflammatory agent.

According to another aspect, there is provided a method of releasing at least one active agent within a subject, the method comprising providing and inserting the medical device disclosed herein into a patient, thereby sustainably release the active agent proximal to the medical device.

According to another aspect, there is provided a method of reducing the risk of intubation-associated disorders or injuries, the method comprising providing and inserting the medical device disclosed herein into a patient, the medical device being in a form of a tube, thereby sustainably release the active agent proximal to the tube.

According to another aspect, there is provided a method of forming the medical device disclosed herein, comprising: (a) providing a polymeric solution comprising at least one active agent; (b) electrospinning the polymeric solution on at least a portion of the medical device to thereby produce a medical device at least partially coated by a composition comprising an electrospun biodegradable nanofiber and at least one active agent.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-B. Image of the adhesion testing setup. Two adjunct ETTs (25 mm) were coated by a layer of electrospun fibers having mometasone furoate (MF) encapsulated therein (MF3; 20% PLGA+3% MF) (FIG. 1A). A non-limiting illustration of an exemplary ETT, partially coated by the electrospun fibers of the invention (FIG. 1B).

FIGS. 2A-B. Calibration curve of MF in pure methanol (FIG. 2A) and 1% SDS aqueous solution: methanol (1:3) (FIG. 2B) using UV-Vis at 248 nm.

FIGS. 3A-E. Morphology of electrospun PLGA fibers loaded with different concentrations of MF, as evaluated by SEM. 20% PLGA (FIG. 3A), MF1 (20% PLGA+1% MF) (FIG. 3B), MF3 (20% PLGA+3% MF). Scale bar=20 μm (FIG. 3C), and Image of a MF1-coated tube (right end) (FIG. 3D), and flow curves of viscosity of the spinning solutions containing 20% PLGA: blank, 1%, and 3% MF (FIG. 3E).

FIGS. 4A-B. Size and orientation of electrospun PLGA fibers loaded with different concentrations of MF. Diameter distribution (FIG. 4A) and orientation of fibers presented on coated ETT (FIG. 4B).

FIGS. 5A-D present SEM micrographs of the morphology of the electrospun 20% PLGA+3% MF fibers during incubation in PBS at 37° C. dry (FIG. 5A), after 0 min (FIG. 5B), after 2 h (FIG. 5C), after 24 h (FIG. 5D). Scale bar=10 μm.

FIGS. 6A-B. Tensile strength of MF3 fiber mats after incubation in PBS. Stress vs. strain tensile test (FIG. 6A), and calculated elastic modulus (FIG. 6B). (n=4 for each time point).

FIGS. 7A-B. Adhesion strength between MF3 and ETT before and during incubation in PBS bath at 37° C. Force-strain tensile test (FIG. 7A) and maximal recorded adhesion force; (n=4 per time point) (FIG. 7B).

FIG. 8. Cumulative percentage of MF released from MF1 fibers into 1% SDS aqueous solution at 37° C., over 14 days.

FIGS. 9A-B present graphs showing drug release data fitted to kinetic models of (FIG. 9A) Higuchi and Korsmeyer-Peppas (FIG. 9B).

FIGS. 10A-B. Stability of MF fibers upon storage. The weight of MF-coated tubes during storage (FIG. 10A). Drug loading at the beginning and end of the stability study (FIG. 10B).

FIGS. 11A-D. Laryngeal and tracheal damage evaluation in animals intubated with ETT or MF1-coated ETT, or with no tube (control). Laryngeal mucosal thickness (FIG. 11A), laryngeal edema (FIG. 11B), tracheal mucosal thickness (FIG. 11C), and tracheal edema (FIG. 11D). Values are mean±SD. ANOVA P<0.01 (FIG. A, B) and Tukey's P values.

FIGS. 12A-E. Representative histological sections of mucosal thickness (subglottic level): Control (non-intubated) (FIG. 12A), Intubated rat (ETT) (FIG. 12B), Intubated rat (MF1-coated ETT) (FIG. 12C) (Magnification ×100). Representative histological sections of submucosal glands (subglottic level) of ETT-intubated rat (ETT) (FIG. 12D), and an MF1-coated ETT-intubated rat (FIG. 12E). Arrows indicate the edema (magnification ×100).

### DETAILED DESCRIPTION OF THE INVENTION

The present invention provides compositions comprising an electrospun biodegradable nanofibers and at least one active therapeutic agent fixed or encapsulated therein. The electrospun biodegradable nanofiber may form a polymeric matrix serving as coatings for medical devices, such as medical tubes, so to locally and sustainably release the incorporated active agent. The present invention further provides methods of locally and sustainably releasing an active agent from a medical device, such as tubes. The invention further provides methods of coating a medical device with a coating with the disclosed compositions.

There is significant morbidity associated with the use of medical devices such as tubes (e.g., ETT). Systemic steroids administration remains the mainstay of treatment for post-extubation complications; however, possible significant adverse effects and inadequate efficacy limit their use. As demonstrated herein, local delivery of an active agent (e.g., mometasone furoate; MF) utilizing a coated ETT possessing specific mechanical features, was shown to be effective for decreasing laryngeal mucosal thickness and submucosal gland edema. The MF-coated ETT provided sustained release of MF for prolonged time (e.g., over 14 days). Mechanical stability of the coating and good adhesiveness to the tube was observed after intubation and extubation and after storage for different time periods.

As demonstrated herein, electrospun fibers are advantageous for forming a coating for a medical-device by virtue of their relatively small diameters, unique physical and mechanical properties, large surface area to volume ratio, which improves the solubility of additional agents (e.g., drugs), and the capability to act as a drug reservoir, and modulate the release profile of the agent.

The present invention is based, in part, on the finding that electrospun fibers can be used as a coating for medical device so to locally release a pharmaceutical agent (e.g., steroids) under a controlled manner. As demonstrated herein below, a steroid-eluting ETT that releases a steroid by a controlled manner locally to the laryngeal and tracheal lumen, was developed.

As used herein, the term “controlled manner” refers to control of the rate and/or quantity of an agent released by the coatings of the invention. The controlled release can be continuous or discontinuous, and/or linear or non-linear. This can be accomplished using one or more types of polymer compositions, drug loadings, inclusion of excipients or degradation enhancers, or other modifiers, administered alone, in combination or sequentially to produce the desired effect.

According to some embodiments, there is provided a medical device at least partially coated by a composition comprising an electrospun biodegradable nanofiber and at least one active agent, the active agent being encapsulated within the electrospun biodegradable fiber, so to locally and sustainably release the active agent.

In some embodiments, the biodegradable nanofiber comprises a polymer or copolymer selected from a miscible polymer, and an enzymatic-degradable polymer, or other stimuli-responsive polymer.

A miscible polymer, in some embodiments, is a polymer which upon contact with physiological conditions, undergoes degradation for a predetermined period of time, so as to release an active agent encapsulated therein.

For endotracheal tubes, as a non-limiting example, the tubes may be coated with a miscible polymer and/or an enzymatic-degradable polymer which undergoes sustained degradation in contact with the mucosal surfaces (e.g., the subglottic or tracheal mucosa).

In some embodiments, the fiber is characterized by a desired adhesion force. In some embodiments, the term “adhesion force” in the context of the present invention is understood to mean the force that occurs between the fiber and the exterior surface of the medical device, causing adhesion of the two substances to each other. This force may also refer to as “surface adhesion” or simply “adhesion”. In some embodiments, the desired adhesion force is from 0.4 to 0.9N, or in some embodiments, from 0.5N to 0.7N. In some embodiments, the adhesion force is 0.4N, 0.5N, 0.6N, 0.7N, or 0.8N, including any value and range there between.

In some embodiments, the fiber is characterized by a desired Young's Modulus. In some embodiments, the term “Young's modulus” (which is also referred to as “the modulus of elasticity”, “elastic modulus”, or “tensile modulus”) denotes a modulus of elasticity describing a property or parameter which is equal to a ratio between a mechanical tension and a corresponding elongation.

In some embodiments, the value of Young's modulus may be temperature-dependent. In some embodiments, the value of Young's modulus refers to the value as measured at room temperature (e.g., about 25° C.). In some embodiments, the fiber has a Young's Modulus in the range of 10-145 MPa. In some embodiments, the fiber has a Young's Modulus in the range of 12-18 MPa. In some embodiments, the fiber has a Young's Modulus of 10 MPa, 20 MPa, 30 MPa, 45 MPa, 55 MPa, 75 MPa, 95 MPa, 110 MPa, 120 MPa, 130 MPa, or 140 MPa, including any value and range there between.

In some embodiments, the electrospun fiber is characterized by a desired tensile strength. In some embodiments, the term “tensile strength”, as used herein, relates to the maximum stress that a material can withstand while being stretched or pulled. In some embodiments, the electrospun fiber is characterized by a tensile strength in a range of 0.2 to 2 MPa. In some embodiments, the electrospun fiber is characterized by a tensile strength in a range of 0.3 to 0.8 MPa. In some embodiments, the electrospun fiber is characterized by a tensile strength of 0.2 MPa, 0.3 MPa, 0.9 MPa, 1.5 MPa, or 2 MPa, including any value and range there between.

In some embodiments, the median size (e.g., of the diameter) of the fibers, ranges from about 100 nanometer (nm) to 2000 nanometers. In some embodiments, the median size ranges from about 200 nanometer to about 2000 nanometers. In some embodiments, the median size ranges from about 500 nanometers to 1500 nanometer.

The term “diameter” as used herein refers not only to the technical geometric term but, in some embodiments, may also refer to the non-technical usage referring to an approximation of the width of the fiber.

In some embodiments, the median size (e.g., the diameter) of the electrospun fibers loaded with the active agent is increased by at least 1%, 5%, 15%, 30%, 35%, 40%, 45%, 50%, 55%, or 60%, comparing to a electrospun fiber lacking the presence of the active agent.

In some embodiments, at least 40%, at least 50%, at least 60%, at least 70%, at least 80%, or at least 90% of electrospun fibers are deposited in a predominantly aligned orientation. The term “predominantly aligned orientation” refers to the fibers being aligned along, or with respect to the main axis of the medical device (e.g., tube). In some embodiment, by “aligned orientation”, it is meant to refer to up to ±5 degrees with respect to the tube main axis.

In some embodiments, the electrospun fiber is characterized by a desired mesh porosity (also referred to as “fibrous mesh porosity”). In some embodiments, the term “mesh porosity” refers to the ratio of pore area to the total area of the fibers mesh. In some embodiments, the fiber is characterized by a mesh porosity of from 78% to 92%. In some embodiments, the fiber is characterized by a mesh porosity of from 80% to 90%. In some embodiments, the fiber is characterized by a mesh porosity of from 78%, 79%, 80%, 81%, 82%, 83%, 84%, 85%, 86%, 87%, 88%, 89%, or 90%, including any value and range there between.

In some embodiments, the pore size is in the range of 5 to 15 μm. In some embodiments, the pore size is in the range of 10 to 12 μm. In some embodiments, the pore size is 5 μm, 6 μm, 7 μm, 8 μm, 9 μm, 10 μm, 11 μm, 12 μm, 13 μm, 14 μm, 15 μm, including any value and range therebetween. In some embodiments, by “pore size” it is meant to refer to a size of at least one dimension of the pore (e.g., the diameter).

In some embodiments, by “coated” it is meant to refer to uniformly coated. In some embodiments, by “uniformly coated” it is meant to refer to a uniform coating having a thickness that varies within less than 30%, less than 20%, or in some embodiments, less than 10%.

In some embodiments, the size of the coating thickness is from 150 μm to 300 μm. In some embodiments, the size of the coating thickness is from 160 μm to 180 μm. In some embodiments, the size of the coating thickness is about 150 μm, 155 μm, 160 μm, 165 μm, 170 μm, 175 μm, 180 μm, 185 μm, or 190 μm, including any value and range there between.

In some embodiments, the encapsulation (or “incorporation”) of the active agent within the fiber is meant that the disclosed bioactive agent is at least 100 μg/cm² fiber.

In some embodiments, the mass ratio of the active agent to the polymer ratio is from 1:20 to 1:5, respectively, e.g., 1:20, 1:15, 1:10, or 1:5, including any value and range there between.

As used herein, an “active agent” is one that produces a local effect in a subject (e.g., an animal). Typically, it is a pharmacologically active substance. The term is used to encompass any substance intended for use in the diagnosis, cure, mitigation, treatment, or prevention of disease or in the enhancement of desirable physical or mental development and conditions in a subject.

Active agents can be synthetic or naturally occurring and include, without limitation, organic and inorganic chemical agents, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like.

Non limiting examples of active agents include anti-inflammatory agents; antimicrobial agents such as antibiotics and antifungal agents; anti-thrombogenic and anticoagulant agents such as heparin, coumadin, protamine, and hirudin; antineoplastic agents and anti-proliferative agents such as etoposide, podophylotoxin; antiplatelet agents including aspirin and dipyridamole; compounds that lower surface tension including surfactant; hormonal agents; nonsteroidal anti-inflammatory drugs (NSAIDs); antimitotics (cytotoxic agents) and antimetabolites such as methotrexate, colchicine, azathioprine, vincristine, vinblastine, fluorouracil, adriamycin, and mutamycinnucleic acids. Anti-inflammatory agents for use in the present invention include but are not limited to glucocorticoids, their salts, and derivatives thereof, such as cortisol, cortisone, fludrocortisone, prednisone, prednisolone, 6α-methylprednisolone, triamcinolone, betamethasone, dexamethasone, beclomethasone, aclomethasone, amcinonide, clebethasol and clocortolone. In exemplary embodiments, the active agent is mometasone furoate.

In some embodiments, a combination of therapeutic agents from the same group, or from other groups are provided (e.g. two cytotoxic agents, or antibiotics and steroids).

In some embodiments, the active agent has a lipophilic nature. Non-limiting lipophilic active agents include comprises one or more of a cannabinoid, alpha tocopherol, amphotericin B, atorvastatin, azithromycin, beclomethasone, budesonide, caspofungin, ciprofloxacin, clemastine, clofazimine, cyclosporine, dihydroergotamine, dronabinol, dutasteride, erythromycin, felodipine, fentanyl, flecainide, fluticasone furoate, fluticasone propionate, furosemide, glycopyrronium, indacaterol, itraconazole, loxapine, mometasone, nimodipine, tacrolimus, tretinoin, vilanterol, or derivatives or analogues thereof.

In some embodiments, the disclosed composition may allow sustained release of the active agent into a physiological medium. In some embodiments, the term “sustained release” means control of the rate of dissolution of the active agent in a body fluid or medium such that it is slower than the intrinsic dissolution rate of the active agent in such a medium, and allows prolonged drug exposure.

In some embodiments, the release of the active agent is triggered by a physiological trigger, e.g., a physiological condition in a body. Exemplary physiological triggers are, without being limited thereto, pH, enzymes, and temperature.

The invention is not limited by the nature of the medical device; rather, any medical device can include the electrospun biodegradable coating described herein. Thus, as used herein, the term “medical device” refers generally to any device that has surfaces that can, in the ordinary course of their use and operation, contact bodily tissue, organs or fluids such as saliva or blood. Examples of medical devices include, without limitation tubes, such as, endotracheal tubes, tracheal tubes, ear ventilation tubes, intrauterine device and cochlear implants.

The duration and quantity of the release of the active agent can be programmed at the time of the coating. For endotracheal tubes, the tubes are typically coated in those parts of the tube that will be in contact with the mucosal surfaces, and that part around the inflatable cuff in contact with the subglottic or tracheal mucosa. Those are the sites where inflammation and granulation tissue typically occur. The typical distance of the preferably coated area of the ETT is the distal half of the tube.

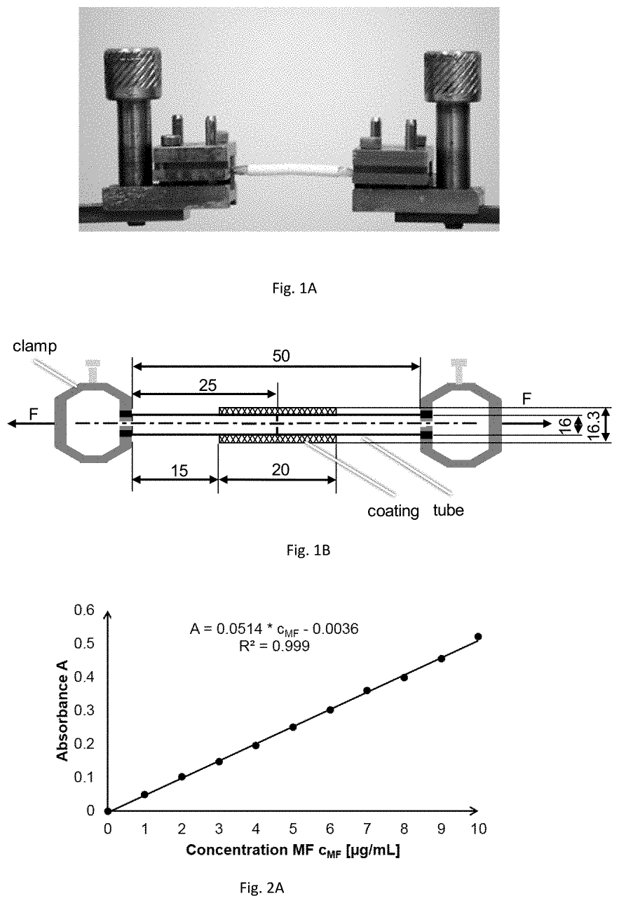

Reference is made to FIG. 1B, a non-limiting illustration of a medical tube (e.g., an ETT) partially coated by the electrospun fibers as disclosed herein. As illustrated, an ETT may be coated in the distal half of the tube, such as to release the agent at the suspected inflammation sites and granulation tissue.

### Electrospun Fibers

According to some embodiments, the compositions of the invention comprise at least one type of electrospun nanofiber and at least one agent encapsulated therein.

In some embodiments, the electrospun nanofiber comprises a biodegradable polymer. As used herein, the term “biodegradable” refers to materials which are enzymatically or chemically or otherwise degraded in vivo into simpler chemical species.

In some embodiments, the electrospun nanofiber comprises a hydrolysable polymer. In some embodiments, by “hydrolysable polymer” it is meant to refer to polymer which undergoes hydrolysis in physiological conditions (e.g., within a body).

In some embodiments, the electrospun nanofiber comprises an enzymatic-degradable polymer. In some embodiments, the electrospun nanofiber comprises a stimuli-responsive polymer.

In some embodiments, or hydrolysable polymers may be made to have slow degradation times and generally degrade by bulk hydrolytic mechanisms.

In some embodiments, degradation time of the polymer would be at least 3 h, 6 h, 12 h, 18 h, 24 h, 1 day, 2 days, 3 days, 5 days, 10 days, or 30 days including any value and range therebetween.

In some embodiments, by “degradation time of the polymer” it is meant to refer to the time range in which the polymeric material start to lose from its original mass, till to lose of 50% of its original mass.

In some embodiments, by “degradation time of the polymer” it is meant to refer to the time over which a wet polymeric material would lose at least 10% of its tensile strength.

In some embodiments, the biodegradable polymer is selected from, without being limited thereto, polylactic acid (PLA), polyglycolic acid (PGA), polycaprolactone (PCL), poly(Lactide-co-Glycolide) (PLGA), polydioxanone (PDO), trimethylene carbonate (TMC), polyethyleneglycol (PEG) and a combination of same.

In some embodiments, the biodegradable polymer is selected from, without being limited thereto. polymers and copolymers of vinyl monomers including polyvinyl alcohols, polyvinyl ketones, polyvinylcarbazoles, polyvinyl esters such as polyvinyl acetates, polyvinyl halides such as polyvinyl chlorides, ethylene-vinyl acetate copolymers (EVA), polyvinylidene chlorides, polyvinyl ethers such as polyvinyl methyl ethers, polyvinylpyrrolidone, vinyl aromatics such as polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystyrene (SEBS) copolymer), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097); silicone polymers and copolymers; poly(carboxylic acid) polymers and copolymers including polyacrylic and polymethacrylic acid, and salts thereof, ethylene-methacrylic acid copolymers and ethylene-acrylic acid copolymers, where some of the acid groups can be neutralized with either zinc or sodium ions (commonly known as ionomers); acrylate and methacrylate polymers and copolymers (e.g., n-butyl methacrylate); acetal polymers and copolymers; cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polyamide polymers and copolymers including nylon 6,6, nylon 12, polycaprolactams, polyacrylamides and polyether block amides; polysulfone polymers and copolymers including polyarylsulfones and polyethersulfones; resins including alkyd resins, phenolic resins, urea resins, melamine resins, epoxy resins, allyl resins and epoxide resins; polycarbonates; polyacrylonitriles; polybenzimidazoles; polyesters including polyethylene terephthalates and aliphatic polyester polymers and copolymers of alpha-hydroxy acids such as polylactide (including d-, l- and meso forms), polyglycolide and poly(lactide-co-glycolide), epsilon-caprolactone, poly(lactide-co-caprolactone), polyhydroxybutyrate, polyhydroxyvalerate, poly(para-dioxanone), polymers of trimethylene carbonate (and its alkyl derivatives), 1,4-dioxepan-2-one, 1,5-dioxepan-2-one, and 6,6-dimethyl-1,4-dioxan-2-one; polyether polymers and copolymers including polyarylethers such as polyphenylene ethers, polyether ketones, polyether ether ketones, and polyalkyl oxides such as polyethylene oxide (PEO) and polypropylene oxide; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; waxes, such as paraffin wax; biopolymers, such as polypeptides, proteins and polysaccharides and fatty acids (and esters thereof), including collagen, dextranomer fibrin, fibrinogen, elastin, chitosan, gelatin, starch, glycosaminoglycans such as hyaluronic acid.

In another embodiment, said composition has a porosity span of at least 30%, at least 35%, at least 40%, at least 45%, at least 50%, at least 55%, at least 60%, at least 65%, at least 70%, at least 75%, at least 80%, at least 85%, at least 90% or at least 95%. In another embodiment, said porosity comprises a plurality of interconnected tunnels within said composition. In another embodiment, the composition comprises pores having a pore size ranging from 0.1 to 100 micrometer.

In another embodiment, the composition comprises a plurality of electrospun nanofibers types and plurality agents, wherein each type of electrospun nanofiber comprises at least one type of agent.

The term “electrospun” or “(electro)sprayed” when used in reference to polymers are recognized by persons of ordinary skill in the art and includes fibers produced by the respective processes. Such processes are described in more detail infra.

Methods for manufacturing electrospun elements as well as encapsulating or attaching molecules thereto are disclosed, inter alia, in WO 2014/006621, WO 2013/172788, WO 2012/014205, WO 2009/150644, WO 2009/104176, WO 2009/104175, WO 2008/093341 and WO 2008/041183.

Manufacturing of electrospun elements may be done by an electrospinning process which is well known in the art. Following is a non-limiting description of an electrospinning process. One or more liquefied polymers (i.e., a polymer in a liquid form such as a melted or dissolved polymer) are dispensed from a dispenser within an electrostatic field in a direction of a rotating collector. The dispenser can be, for example, a syringe with a metal needle or a bath provided with one or more capillary apertures from which the liquefied polymer(s) can be extruded, e.g., under the action of hydrostatic pressure, mechanical pressure, air pressure and high voltage.

The rotating collector (e.g., a drum) serves for collecting the electrospun element thereupon. Typically, but not obligatorily, the collector has a cylindrical shape. The dispenser (e.g., a syringe with metalic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Inverse electrostatic configurations for establishing motions of negatively charged jet from the dispenser to the collector are also contemplated.

At a critical voltage, the charge repulsion begins to overcome the surface tension of the liquid drop. The charged jets depart from the dispenser and travel within the electrostatic field towards the collector. Moving with high velocity in the inter-electrode space, the jet stretches, and solvent therein evaporates, thus forming fibers which are collected on the collector, thus forming the electrospun element.

Non-limiting examples of processes for electrospinning drug-loaded fiber coating for tubes (e.g., ETT) include use of a rotating mandrel. The tube may be assembled on a wire (e.g., stainless-steel wire) such as with a diameter of 1.2 mm, functioning as the grounded collector. A spin dope is formed, e.g., by dissolving the polymer (e.g., PLGA) in THF/DMF (4:1) at a concentration of 20%, and then the agent may be added in a desired polymer/agent ratio (e.g., 0.1:20-5:20). A syringe pump may be used to pump the spin dope through a needle (e.g., gauge G25) with a flow rate of about 0.3 mL/h. The distance to the collector may be about 8 cm, the applied voltage may be about 9 kV, resulting in an electrical field of about 1.125 kV/cm. Each tube was coated for 12 min. The radial velocity of the mandrel may result in a tangential velocity of the tube of 0.047 m/s. The process may be carried out at ambient conditions with a measured humidity of about 45%.

As used herein, the phrase “electrospun element” refers to an element of any shape including, without limitation, a planar shape and a tubular shape, made of one or more non-woven polymer fiber(s), produced by a process of electrospinning. When the electrospun element is made of a single fiber, the fiber is folded thereupon, hence can be viewed as a plurality of connected fibers. It is to be understood that a more detailed reference to a plurality of fibers is not intended to limit the scope of the present invention to such particular case. Thus, unless otherwise defined, any reference herein to a “plurality of fibers” applies also to a single fiber and vice versa. In some embodiments, the electrospun element is an electrospun fiber, such as electrospun nanofiber. As used herein the phrase “electrospun fiber” relates to a fibers formed by the process of electro spinning.

One of ordinary skill in the art will know how to distinguish an electrospun object from objects made by means which do not comprise electrospinning by the high orientation of the macromolecules, the fiber morphology, and the typical dimensions of the fibers which are unique to electrospinning.

The electrospun fiber may have a length which is from about 0.1 millimeter (mm) to about 20 centimeter (cm), e.g., from about 1-20 cm, e.g., from about 1-10 cm. According to some embodiments of the invention, the length (L) of the electrospun fibers of some embodiments of the invention can be several orders of magnitude higher (e.g., 10 times, 100 times, 1000 times, 10,000 times, e.g., 50,000 times) than the fiber's diameter (D).

Laboratory equipment for electrospinning can include, for example, a spinneret (e.g. a syringe needle) connected to a high-voltage (5 to 50 kV) direct current power supply, a syringe pump, and a grounded collector. A solution such as a polymer solution, sol-gel, particulate suspension or melt is loaded into the syringe and this liquid is extruded from the needle tip at a constant rate (e.g. by a syringe pump).

In some embodiments, parameters of the electrospinning process may affect the resultant substrate (e.g. the thickness, porosity, etc.). Such parameters may include, for example, molecular weight, molecular weight distribution and architecture (branched, linear etc.) of the polymer, solution properties (viscosity, conductivity & and surface tension), electric potential, flow rate, concentration, distance between the capillary and collection screen, ambient parameters (temperature, humidity and air velocity in the chamber) and the motion and speed of the grounded collector. Accordingly, in some embodiments, the method of producing a substrate as described herein includes adjusting one or more of these parameters.

General:

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Materials and Methods

Mometasone Furoate (MF) (≥98%), PLGA (85:15) LACTEL® B6001-1, and Phosphate buffered saline (PBS) powder were purchased from Sigma-Aldrich (Rehovot, Israel). The solvents Tetrahydrofuran (THF), Dimethylformamide (DMF), Methanol (MeOH), and Ethanol (EtOH) were obtained from Bio-Lab ltd. (Jerusalem, Israel). Acetone was purchased from Gadot Biochemical Industries ltd. (Haifa, Israel).

Closed suction system for endotracheal tubes (ETT) KimVent* for neonates/pediatrics were purchased from Kimberly-Clark LLC (Roswell, Giorgia, USA) and from Halyard Health (Alpharetta, USA). The inner diameter of the tubes was 1 mm, the outer diameter was 1.6 mm.

Preparation of Polymeric Nanofibers Encapsulated with Steroid

Electrospinning of drug-loaded fiber mats for coating endotracheal tubes (ETT) was performed using a rotating mandrel. PLGA was dissolved in THF/DMF (4:1) at a concentration of 20%, and then MF was added in a polymer/drug ratio of either 1:20 (MF1) or 3:20 (MF3).

ETTs were cut to a length of 30 mm and assembled on a stainless-steel wire with a diameter of 1.2 mm, functioning as the grounded collector. A syringe pump (Harvard Apparatus, Holliston, USA) was used to pump the spin dope through a needle (gauge G25) with a flow rate of 0.3 mL/h. The distance to the collector was 8 cm, the applied voltage was 9 kV, resulting in an electrical field of 1.125 kV/cm. Each tube was coated for 12 min. The radial velocity of the mandrel resulted in a tangential velocity of the tube of 0.047 m/s. The process was carried out at ambient conditions with a measured humidity of about 45%.

Rheological Properties of Spinning Solutions

The effect of MF addition on the viscosity of the spinning solution was determined. For this purpose, 20% PLGA, 20% PLGA+1% MF and 20% PLGA+3% MF were each dissolved in THF/DMF (4:1) and stirred with a magnetic stirrer for 48 h. The viscosity measurements were performed at room temperature using a rheometer (Discovery HR-2 rheometer by TA instruments, New Castle, USA). Experiments were carried out using 0.5 mL of spinning solution, tested in a cone-plate configuration with a distance of 32 μm between cone and plate. Viscosities of the solutions were measured over a series of shear rates starting from 0.56 1/s up to 100 1/s.

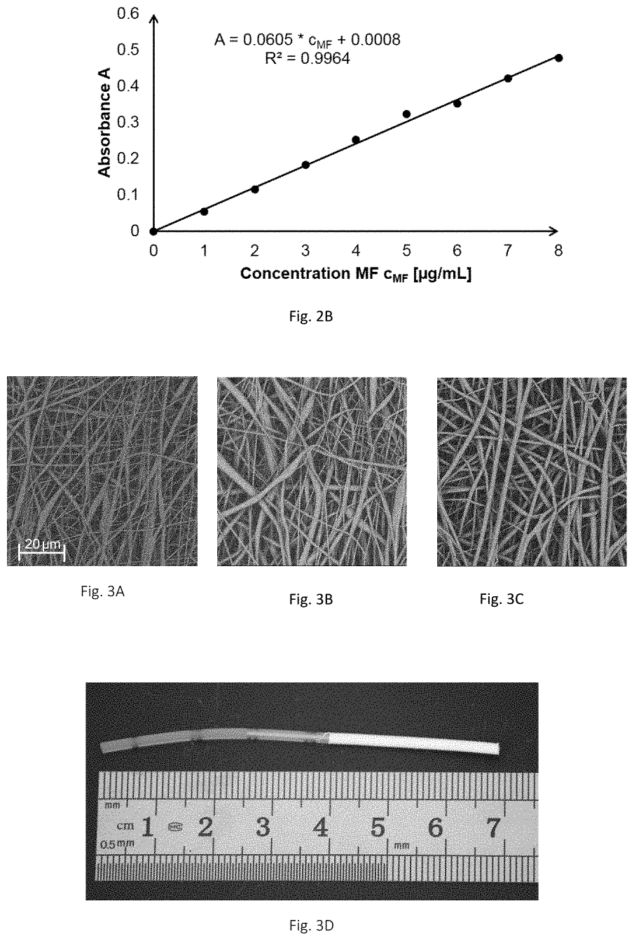

The three tested polymer solutions are constant over the applied shear rate and hence show Newtonian behavior (FIG. 3E). Hereby an increase of the dynamic viscosity can be observed with increasing concentration of MF in the polymer solutions. This was expected due to the increasing ratio of solutes to solvent with growing amount of MF in the solution.

Characterization of Fibers

Morphology, Size and Orientation of Fibers

Scanning Electron Microscopy (SEM) was used in order to investigate the morphologies and sizes of the fibers by Phenom SEM (FEI Company, Hillsboro, Oreg., USA). For this purpose, fiber-coated ETT were cut to a length of 0.5 cm and gold sputtered before microscopy. Image analysis was performed using ImageJ software (National Institutes of Health, Bethesda, Md., USA) to determine the distributions of fiber diameters and orientation from the SEM micrographs. For each group of samples, the diameters of at least 80 fibers were measured prior to taking an average value.

Degradation of Fiber Mats

Observation of the degradation of fiber mats was done after placing the mats (10 mm×5 mm) into 0.01 M PBS (pH=7.4) media and storing the specimen in an incubator at 37° C. and 50 rpm. At predefined time intervals (0 min., 2 h, 24 h) the samples were taken out of the media and prepared for SEM imaging.

Dimensional Stability of Fiber Mats.

The dimensional changes of fiber mats under physiological conditions was observed using 0.01 M PBS solution (pH=7.4) at 37° C. For this purpose, the electrospun mats of MF3 were cut into pieces of 10 mm×5 mm samples (n=3 per type per time point). Engineering strain was determined by considering the length, width and thickness of the specimens at dry state and after placement into PBS solution for 1 min, 2 h and 24 h in the media.

Mechanical Properties

Tensile Testing

Tensile tests of aligned fibers mats were carried at force-controlled mode using a horizontal tensile machine equipped with a temperature controlled water bath and load cell with 1 mN resolution (Model 31/1435-03, Sensotec, Columbus, Ohio). Fiber mats made of MF3 in 4:1 THF/DMF were cut into samples of 20 mm×2 mm, and average thickness of 179±4 um (n=4 per type per time point). The length between the clamps was set to 10 mm and the strain rate was 0.05 1/s. Tensile strength of fiber mats was measured under dry conditions and immersed in equivalent PBS media at 37° C., after storage for 0 h, 2 h and 24 h in 0.01 M PBS (pH=7.4) at 37° C. in a rotary incubator (50 rpm).

Adhesion of Fiber Mat to ETT

The measurement of the adhesion strength between the fiber coating and the ETT was conducted using the aforementioned horizontal tensile machine with similar parameters at force-controlled mode. Sample was prepared by resected an ETT (50 mm long) and then located the two remaining ends (25 mm long) facing each other on the mandrel. Electrospinning directly on the mandrel, as described above, resulted in coated ETT over a total length of 20 mm (each tube section covered along 10 mm). Coating of the specimen was carried out using MF3 in 4:1 THF/DMF. A scheme of the specimen's dimensions is shown in FIG. 1A and FIG. 1B. To measure the adhesivity of the fibers to the ETT, a tensile test procedure was adopted (see above) and the force was registered vs. the strain until slipping of the coating was observed. The adhesion force was measured at dry state, and wet after placing into 0.01 M PBS solution (pH=7.4) at 37° C. for 0 min, 2 (n=4 per type per time point). Testing of the wet samples was performed using the bath setup filled with 0.01 M PBS solution (pH=7.4).

Determination of Drug Loading

A layer of the electrospun fibers mat, part of the tube's coating (˜1 cm in length), was peeled off, weighted and dissolved in acetone. After vortex for 30 seconds, ethanol was added to precipitate PLGA, and the mixture was vortexed and centrifuged for 2 min at 14000 rpm. The supernatant was transferred to a glass vial, and the solvents were evaporated in a vacuum oven at 50° C. for 60 min. The residue was dissolved in methanol, and the amount of MF was determined by UV-Vis spectrophotometer (UV-1800, Shimadzu, Japan) at the wavelength of maximal absorbance of MF at λmax=248 nm (triplicate). The concentrations of the calibration curve ranged from 0-10 μg/mL (FIGS. 2A-B).

In Vitro Drug Release Kinetic Experiments

Tubes (ETT) of 1 cm length coated with electrospun fibers (MF1) were placed in glass vials containing 10 mL of 1% SDS aqueous solution (n=3 per time point), and kept in a rotary (50 rpm) incubator at 37° C. At predetermined time intervals, 1 mL of the medium was discarded. Subsequently the glass vials were refilled with 1 mL of fresh release medium, in order to maintain constant conditions. The release medium was replaced after eight hours, and then after each time point by fresh 1% SDS aqueous solution. For the quantification of MF, the withdrawn 1 mL was diluted using methanol and the quantity of MF was determined by UV-Vis spectrophotometer (UV-1800, Shimadzu, Japan) at wavelength of 248 nm (FIG. 2A-B).

In-Vivo Study

For the purpose of the in vivo study, an animal model based on trans-oral intubation of rats was developed. This model simulates the common daily human clinical scenario of trans-oral intubation, which may result in significant airway morbidity as described above. The animal study was carried out by blindly assigning the rats into the treatment groups

Animals and Study Design

All animal experiments were conducted according to the institutional animal ethical committee guidelines, which conforms to the Guide for the Care and Use of Laboratory Animals published by the US National Institutes of Health (Eighth edition, 2011). In this research, 350-400 gr. male Sprague-Dawley were used. The animals were maintained at the institutional Experimental Surgical Unit, and were fed on normal rodent chow diet unless specified otherwise, with tap water ad libitum. The rats were housed at a constant temperature and relative humidity under a regular light/dark schedule (12:12).

Animals were randomly assigned to one of the 3 experimental groups: non-intubated (control, n=9), intubated rats with blank ETT (n=4), and 1% MF-coated ETT (n=5). Under anesthesia with a combination of 87 mg/kg ketamine and 13 mg/kg xylazine, each rat was intubated trans-orally under direct vision with a 7 cm tube, either blank or MF-coated. Subsequently, the animals were left to breathe through the tube for 3-6 hours and then were extubated. Anesthesia was maintained by addition of the same ketamine xylazine mixture every 45-60 min.

Euthanasia and Organs Harvesting

24 hours post intubations the animals were euthanized by ketamine xylazine mixture over-dose and the larynx as well as a fair segment of the trachea was excised. The excised laryngo-tracheal complex was sectioned into 4 pieces and preserved in 4% paraformaldehyde for histology.

Histology

Paraffin embedded laryngo-tracheal pieces were sectioned by microtome into 5 μm slides which were stained with hematoxylin and eosin (H&E) stain. The sections were photographed and analyzed.

Histopathologic Damage Grading

Histological specimens of the vocal cords, subglottic area, upper trachea and lower trachea were analyzed for the following parameters: Mucosal thickness and Sub-mucosal glands hypertrophy.

Mucosal thickness: For each slide, calculation of mucosal thickness was performed in four areas of the slides then an average thickness was calculated for each slide. Thickness was calculated at each set area of each slide only if the local anatomy seemed preserved. Areas with distorted anatomy were not included in the calculation of the average mucosal thickness.

Sub-mucosal glands hypertrophy: Estimation of sub-mucosal glands hypertrophy was performed for each histological specimen according to a score of 0/1/2 which stands for minimal glands hypertrophy (“0”), glands hypertrophy in <50% of the specimen sub-mucosal area (“1”) and glands hypertrophy in >50% of the specimen sub-mucosal area (“2”).

For analysis purposes, the two laryngeal subsites (vocal cords and subglottic levels) and the two tracheal subsites (upper and lower trachea) were grouped together so the final statistical analysis was performed for the larynx and trachea.

Statistical Analysis

One-way ANOVA and Tukey's multiple comparisons tests were performed in order to examine the significance of the differences in the animal study. GraphPad Prism, version 7 (GraphPad Software, Inc., San Diego, Calif.) was used. Differences were considered significant if P<0.05.

### Example 1

### Morphology, Size and Orientation of Fibers

PLGA nanofibers loaded with MF were successfully fabricated, forming uniform coating directly on the ETT. SEM images (FIG. 3A) of 20% PLGA (blank fibers) and MF1 (20% PLGA+1% MF fibers), demonstrate inhomogeneous fibers by means of diameter, and beads formation along the fibers. However, MF3 (20% PLGA+3% MF) fibers were rather uniform, with occasionally appearance of beads apparently due the low viscosity of the spinning solutions (FIG. 3B). Average diameter of blank fibers was 635 nm, and those of MF1 and MF3 fibers were 757 nm and 1069 nm, respectively. The encapsulation yields, and drug loading percentages were satisfactory (Table 1A and Table 1B).

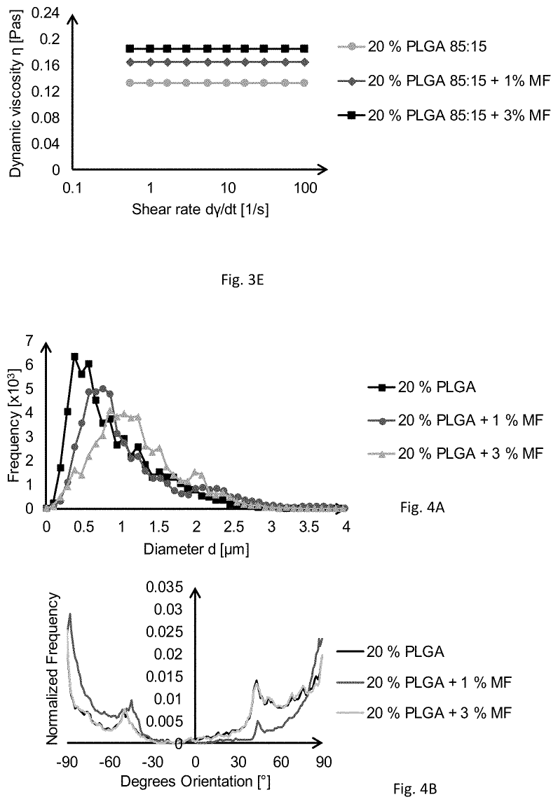

Distributions of the fiber diameter and orientation of fibers are presented in FIG. 4. The effect of the encapsulation of MF on fiber diameters was remarkable, with increasing loading levels of MF; the shear viscosity increased resulting in fiber diameters increased. Due to the relatively low tangential velocity of the rotating mandrel with the ETT, the fibers tend to align along the ETT, as demonstrated in the fiber orientation distribution function (see FIG. 4B) by a pronounced orientation along the main axis of the ETT (+90°/−90° degrees) with minor peaks at −45° and +45°.

### Table 1A-B. Characterization of Fibers

TABLE 1A

| | Fiber diameter | Encapsulation | Drug loading² |
|---|---|---|---|
| Fabrics | (nm) | yield¹ (%) | (wt %) |
| MF-PLGA | 635 ± 396 | — | — |
| MF1-PLGA + 1% MF | 757 ± 322 | 90.17 ± 16.31 | 4.5 ± 0.82 |
| MF3-PLGA + 3% MF | 1069 ± 496 | 80.43 ± 14.37 | 12.1 ± 2.16 |

¹Values are mean of three measurements ± SD.
²Drug loading (%) = [wt drug (observed)/wt polymer] × 100%

TABLE 1B

| | Theoretical | | Encapsulation |
|---|---|---|---|
| | MF | Observed MF | yield |
| Material | [μg] | [μg]] | [%] |
| PLGA (85:15) ± | 82.61 ± 9.50 | 74.49 ± 5.49 | 90.17 ± 16.31 |
| 1% MF | | | |
| PLGA (85:15) ± | 241.38 ± 33.00 | 194.14 ± 11.94 | 80.43 ± 14.37 |
| 3% MF | | | |

### Example 2

### Degradation and Shrinkage of Fiber Mats

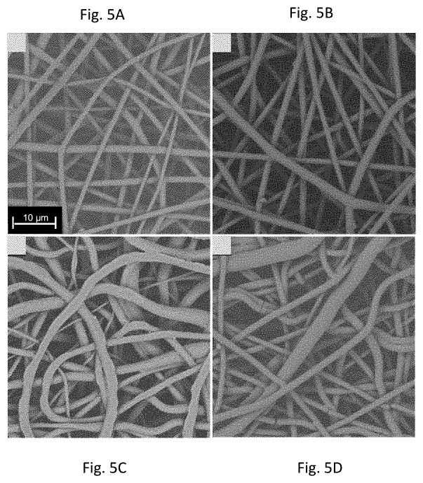

In Table 2, the dimensional changes of MF3 fiber mats are shown. With increasing time in PBS media at 37° C., a decrease in length and width was observed, while the thickness increased from 172.3 μm in the dry state by 72.5% to 296.7 μm after 24 h in PBS (FIG. 5A-D). Without being bound to any particular mechanism, the dimensional change is attributed to the effect of hydration on glass transition temperature (T_{g}). Also, it is suggested that during the electrospinning high elongation of the polymer network results in stretching of polymer macromolecules and consequently in the formation of nanofibers with a high degree of molecular orientation along the fiber main axis. Therefore, enhanced relaxation of stretched polymer macromolecules to their equilibrium is expected across the length of the fibers mat. In addition, typical degradation was observed after 24 h, in which fibers cracked and broke up into shorter fragments.

Table 2 shows dimensional changes of MF3 fiber mats after placement in PBS at 37° C. for different time intervals. The strains along the length, width and thickness of the fibers mat are εₗ, ε_{w}, and εₜ respectively.

TABLE 2

| | | εₗ % | ε_{w} % | εₜ % |
|---|---|---|---|---|
| 0 | h | −0.6 ± 0.66 | −0.6 ± 0.48 | 0.1 ± 0.26 |
| 2 | h | −16.6 ± 0.55 | −18.9 ± 4.31 | 33.4 ± 7.05 |
| 24 | h | −22.9 ± 1.16 | −37.7 ± 2.58 | 72.6 ± 12.12 |

### Example 3

### Mechanical Properties

Tensile Tests

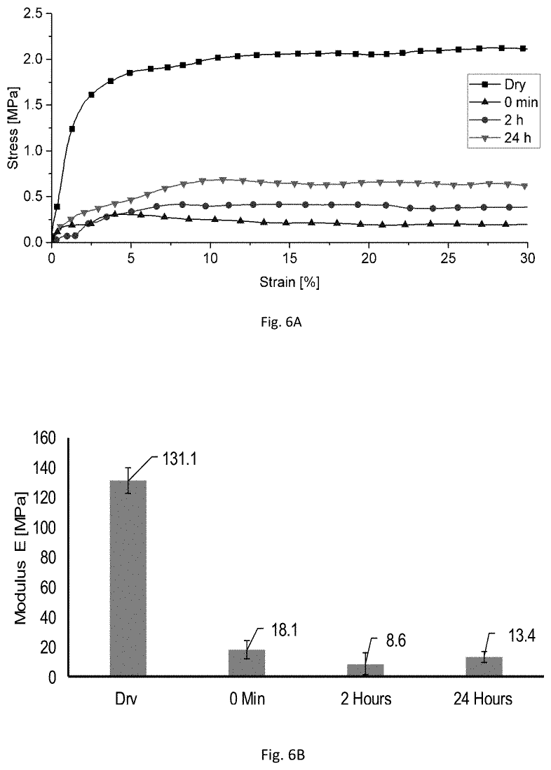

Stress-strain graphs of MF3 fiber mats can be seen in FIG. 6A-B in dry state and wet after predetermined times of degradation. Obviously visible is the qualitatively different behavior of the dry fiber mat compared to the mats tested in PBS bath at 37° C. A significantly higher yield stress as well as ultimate stress was obtained for tensile tests of dry samples in comparison to the wet samples. Consequently, the maximal strain until failure was for the dry PLGA fibers far below the results measured for wet specimens, apparently due to the decrease of the T_{g} in aqueous solution.

In case of samples tested after 0 h and 2 h in media, the ultimate stress as well as the strain at breakdown point could not be detected due to reaching of the limit of the tensile machine at a strain over 325%. While the elastic deformation was in the same range for samples throughout the different time points, the plastic deformation was elevated for the specimens tested in wet conditions. Furthermore, an increase of stress applied during plastic deformation was visible with advancing time of degradation. Also, the strain at failure after 24 h in PBS bath was ˜175%, due to fibers incipient degradation.

Adhesion Test

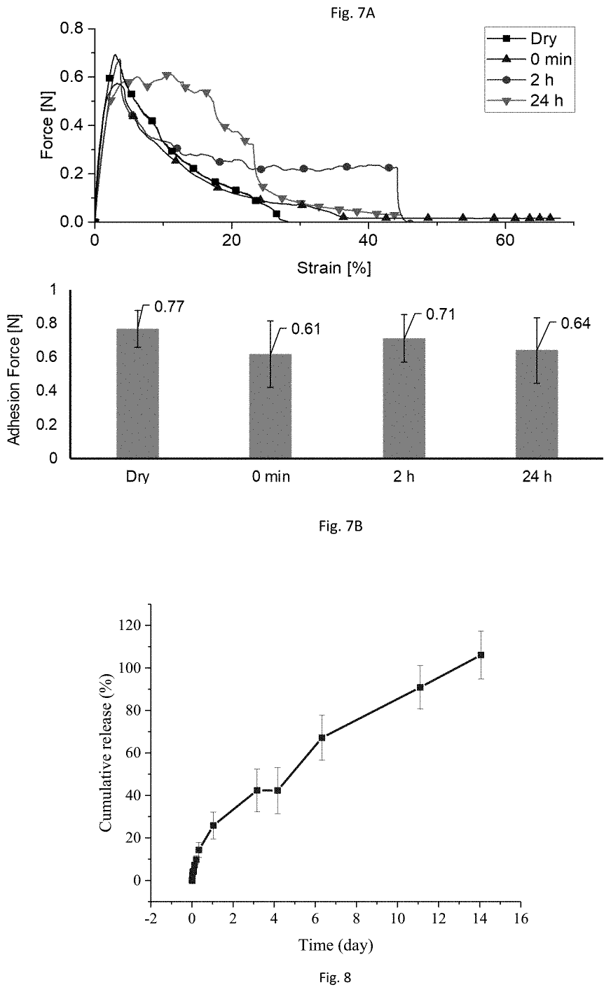

Adhesion between fiber coating and ETT was tested in dry and wet conditions after selected durations of degradation. The results force-strain curve and maximal adhesion force, are shown in FIG. 7. Insignificant changes of the maximal force, were obtained, also after immersion in water for 24 h. Apparently, fibers swelling affects the fibers diameter and fibers mat porosity, thus the effective surface area of the fibers compensate the decrease in the modulus as presented in FIG. 6B.

### Example 4

### In Vitro Drug Release

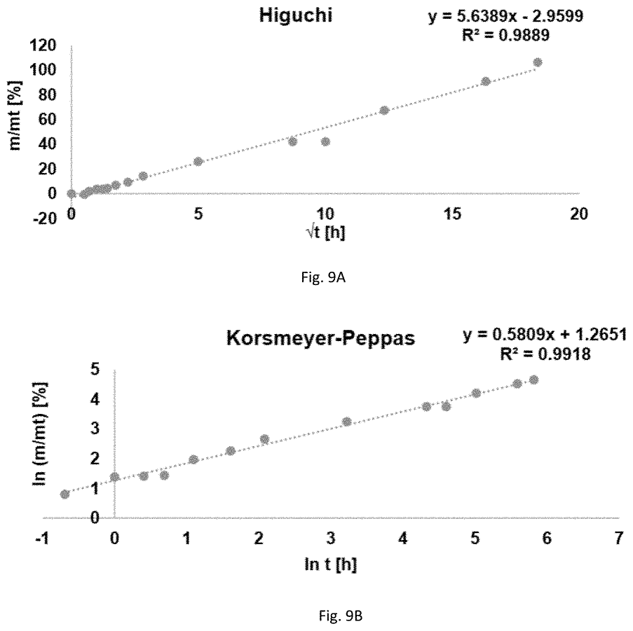

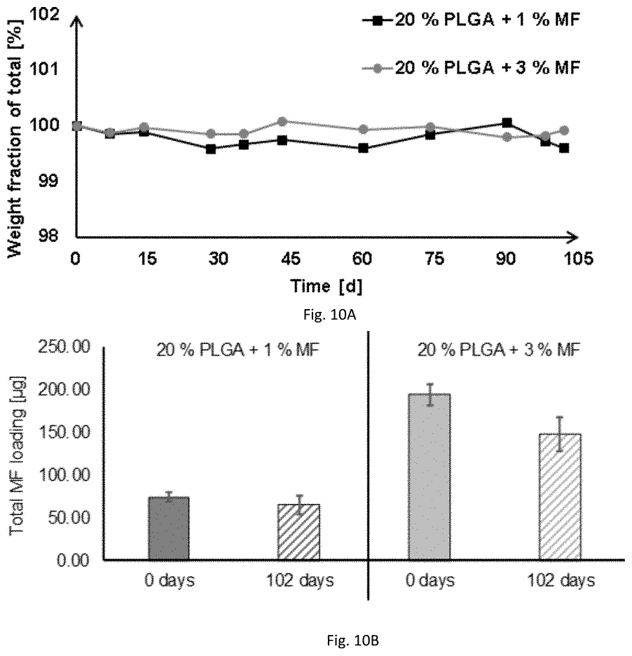

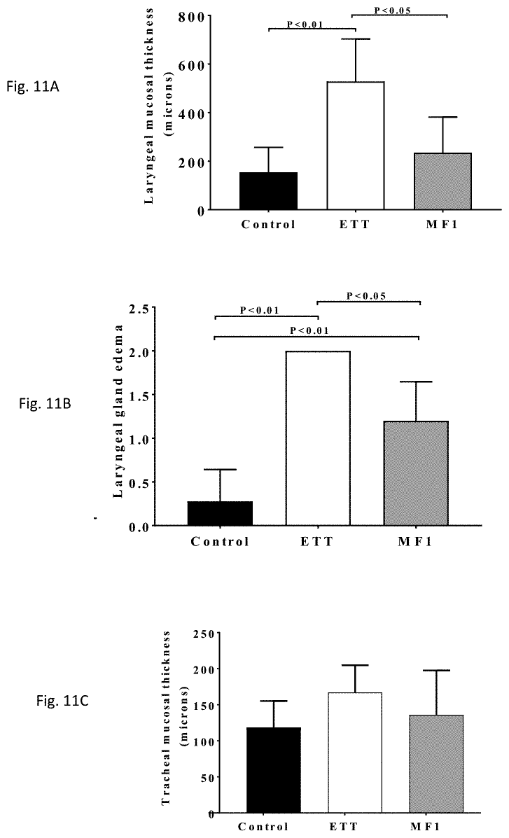

The in vitro release profile of MF from the nanofibers (MF1) was studied in 1% SDS aqueous solution at 37° C. to evaluate the potential application of MF loaded nanofibers as drug delivery system. The cumulative release curve of the drug-loaded nanofibers is shown in FIG. 8. In vitro release profile of MF from coated ETTs exhibited burst effect of approximately 15%, accompanied by a second release phase in a sustained release manner. The cumulative MF released at the end of two weeks was about 100% of the initial drug loading. Diffusion of little amount of MF on or nearby the surface layer of fibers could most likely contribute to the initial burst release. Moreover, different kinetic equations were applied and the best fits with higher correlations were found with Higuchi's law (r²=0.988) and Korsmeyer-Peppas exponential equation (r²=0.991) (FIG. 9). However, while Higuchi model does not take into account the influence of gradual erosion of the matrix, the Korsmeyer-Peppas model allows for the deviation of drug release mechanism from Fick's law and follows an anomalous behavior. Therefore, the Korsmeyer-Peppas equation was used resulting in a value of diffusion exponent, v=0.58 which indicates anomalous transport, due to both phenomena of diffusion and swelling-controlled drug release. These findings are in agreement with the degradation and dimensional change results (FIG. 4).

### Example 5

### In Vivo Study

In this study, animals were intubated for 3-6 h under anesthesia, in order to emulate the damage of intubation in patients. For assessment of intubation damage, histological samples were examined indicating a variable loss of mucosal epithelium, enhanced laryngeal mucosal thickness, and submucosal gland edema. The damage was quantified by measuring the average mucosal thickness in each histology section as well as the degree of submucosal gland edema for the larynx (vocal cords and subglottic levels) and trachea (upper and lower). Scoring of the histological damage was performed by two blinded expert senior pathologists. Herein, the MF1-coated ETT was used for further in vivo efficacy study, due to higher encapsulation yield (Table 1) and better long-term stability upon storage (FIG. 10A-B) compared to MF3, taking into consideration the scale up process, for the development of such a platform in the future. Moreover, due to prescribed time window of 6 h, the use of MF1 (nanofibers) rather than MF3 (microfibers) is favorable, because of faster release rate and higher released amount of the lipophilic drug form PLGA fibers.

The mean laryngeal mucosal thickness of intubated animals using ETT was significantly higher than those of control (non-intubated) and MF1 coated ETT groups (P<0.01), whereas there was no significant difference in tracheal mucosal thickness. Furthermore, the laryngeal submucosal gland hypertrophy of the control group was scored 0.28±0.12, while the ETT was 2, and MF1 coated ETT was 1.2±0.2 (FIG. 7). Again, no significant differences were observed in tracheal specimens between the three groups.

Previous animal models that focused on laryngo-tracheal morbidity have used similar histological criteria as the above for damage assessment including mucosal edema and submucosal glands hypertrophy. In some models a decision was made to worsen airway tissue reaction by twisting the ETT while in situ every selected time interval or by using the largest possible tube diameter.

It was decided, in this animal model, to simulate the standard clinical scenario and not to manipulate the tube or use a large tube. This probably results in less traumatic damage and less mucosal inflammation, although better simulates the typical clinical scenario in patients.

The larynx (which includes the levels of the vocal cords and subglottic area) is known to have the smallest diameter in the human upper airway; therefore, most ETT-related morbidity is diagnosed at this level. The results presented herein show that the same is true for rats as laryngeal damage in this animal intubated cohort was clearly more evident compared to tracheal mucosal damage. It is clinically unlikely to demonstrate significant tracheal (rather than laryngeal) damage after a short intubation interval of 3-6 hours as indeed is shown in the results of this animal model. Longer intubation intervals in rats or humans should present with severe tissue reaction and more upper airway complications. A longer intubation model may show improved therapeutic efficacy of MF-coated tubes as longer duration of intubation will worsen airway tissue reaction on one hand and allow for extended release of MF on the other hand.

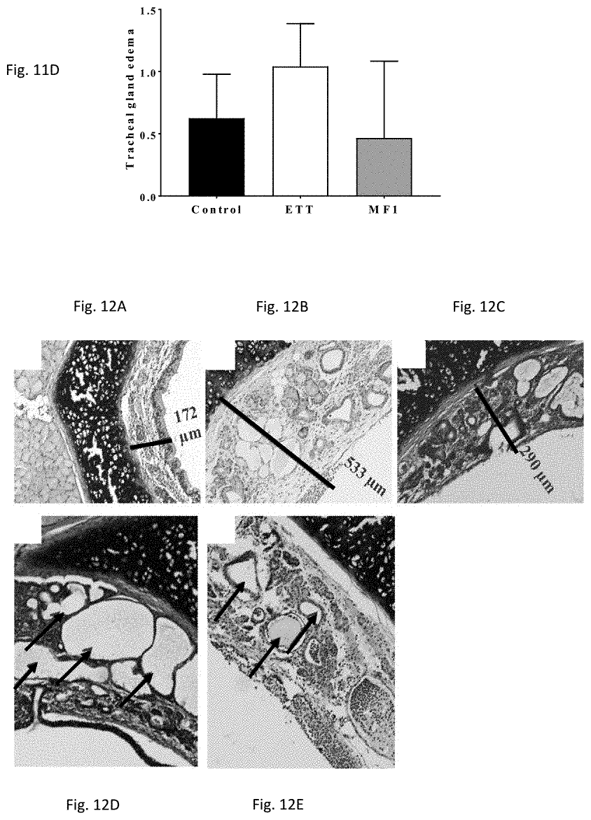

Representative histological sections of mucosal thickness and submucosal glands are depicted in FIG. 12. These findings indicate that there is a therapeutic benefit in vivo of using MF1 coated ETT, by means of maintaining mucosal thickness and reducing edema, taking into consideration that the MF1 coated ETT was placed only for 3-6 h, and since the drug is encapsulated in the polymeric matrix, the release rate of the drug is modified, resulting in the release of small fraction of loaded drug during this time period.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A medical device at least partially coated by a composition comprising an electrospun biodegradable nanofiber and at least one active agent, the active agent being encapsulated within the electrospun biodegradable fiber, so to locally and sustainably release the active agent.

2. The medical device of claim 1, wherein said biodegradable nanofiber comprises a polymer or copolymer selected from a miscible polymer, and an enzymatic-degradable polymer.

3. The medical device of claim 1, wherein said nanofiber is characterized by an adhesion force of 0.4-0.8N to an exterior surface of said medical device.

4. The medical device of claim 1, wherein said nanofiber has a Young's Modulus in the range of 10-145 MPa.

5. The medical device of claim 1, wherein said nanofiber has a tensile strength in a range of 0.2-2 MPa.

6. The medical device of claim 1, wherein said nanofiber has a diameter in the range of 500-1500 nm.

7. The medical device of claim 1, wherein said nanofiber has a Fibrous Mesh porosity of 78-to 92%

8. The medical device of claim 1, wherein said nanofiber has a Fibrous Mesh pore diameter 5-15 μm

9. The medical device of claim 1, wherein said coating comprises a substantially uniform thickness in the range of 150-300 μm.

10. The medical device of claim 1, having an agent-loading capacity of:
(i) 50-500 μg/cm; and/or
(ii) 100-1000 μg/cm² fiber.

11. The medical device of claim 1, wherein the sustainable release of the active agent is for at least 24 hours.

12. The medical device of claim 1, wherein said electrospun nanofiber comprises a polymer selected from the group consisting of poly (lactic-co-glycolic) acid (PLGA), polylactic acid (PLA), polyglycolic acid (PGA), and polycaprolactone (PCL).

13. The medical device of claim 1, wherein the agent is selected from the group consisting of: an anti-inflammatory agent (e.g., steroid), an anti-infective agent (e.g. antibiotics, antifungals), compounds that reduce surface tension (e.g. surfactant), anti-neoplastic agents and anti-proliferative agents, anti-thrombogenic and anticoagulant agents, antiplatelet agents, hormonal agents, nonsteroidal anti-inflammatory drugs (NSAIDs), antimitotics (cytotoxic agents) and antimetabolites.

14. The medical device of claim 1, wherein the medical device is a tracheal tube and the agent is an anti-inflammatory agent.

15. A method of releasing at least one active agent within a subject, the method comprising providing and inserting the medical device of claim 1 into a patient, thereby sustainably release the active agent proximal to the medical device.

16. The medical device of claim 1, for reducing the risk of intubation-associated disorders or injuries.

17. A method of forming the medical device of claim 1, comprising: (a) providing a polymeric solution comprising at least one active agent; (b) electrospinning the polymeric solution on at least a portion of the medical device to thereby produce a medical device at least partially coated by a composition comprising an electrospun biodegradable nanofiber and at least one active agent.
